(19) United States
(12) Patent Application Publication
(10) Pub. No.: US 2025/0122295 A1
(43) Pub. Date: Apr. 17, 2025

(54) METHOD OF TREATMENT OF PHILADELPHIA CHROMOSOME POSITIVE LEUKEMIA

(71) Applicant: CSL LIMITED, Melbourne (AU)

(72) Inventors: Devendra Keshaorao HIWASE, Stonyfell (AU); Timothy Peter HUGHES, Kensington Gardens (AU); Angel Francisco LOPEZ, Medindie (AU); Gino Luigi VAIRO, Northcote (AU)

(73) Assignee: CSL LIMITED, Melbourne (AU)

(21) Appl. No.: 18/775,212

(22) Filed: Jul. 17, 2024

Related U.S. Application Data
(63) Continuation of application No. 18/591,478, filed on Feb. 29, 2024, now abandoned.
Application No. 18/591,478 is a continuation of application No. 17/205,585, filed on Mar. 18, 2021, now abandoned.
Application No. 17/205,585 is a continuation of application No. 16/580,012, filed on Sep. 24, 2019, now abandoned.
Application No. 16/580,012 is a continuation of application No. 15/690,826, filed on Aug. 30, 2017, now abandoned.
Application No. 15/690,826 is a continuation of application No. 15/193,784, filed on Jun. 27, 2016, now abandoned.
Application No. 15/193,784 is a continuation of application No. 14/517,254, filed on Oct. 17, 2014, now abandoned.

(51) Int. Cl. C07K 16/28; A61K 31/122; A61K 31/4545; A61K 31/497; A61K 31/506; A61K 38/17; A61K 38/19; A61K 38/20; A61K 39/395; A61K 45/06; A61K 47/64; A61K 47/68

(52) U.S. Cl. CPC C07K 16/2866; A61K 31/122; A61K 31/4545; A61K 31/497; A61K 31/506; A61K 38/1793; A61K 38/193; A61K 38/202; A61K 39/3955; A61K 39/39558; A61K 45/06; A61K 47/642; A61K 47/6867; C07K 2317/24; C07K 2317/52; C07K 2317/732

(57) ABSTRACT

The invention provides a method for the treatment of Ph+ leukemia in a patient comprising administering to the patient (i) a BCR-ABL tyrosine kinase inhibitor, and (ii) an agent which selectively binds to a cell surface receptor expressed on Ph+ leukemic stem cells. The invention further provides for the use of (i) and (ii) in, or in the manufacture of a medicament for, the treatment of Ph+ leukemia in a patient; and a composition for the treatment of Ph+ leukemia in a patient comprising (i) and (ii); and kits comprising (i) and (ii). In some embodiments, the tyrosine kinase inhibitor is or is not imatinib; or is selected from the group consisting of dasatinib, nilotinib, bosutinib, axitinib, cediranib, crizotinib, damnacanthal, gefitinib, lapatinib, lestaurtinib, neratinib, semaxanib, sunitinib, toceranib, tyrphostins, vandetanib, vatalanib, INNO-406, AP24534, XL228, PHA-739358, MK-0457, SGX393 and DC2036; or is selected from the group consisting of dasatinib and nilotinib. In some embodiments, the agent binds to a receptor involved in signalling by at least one of IL-3, G-CSF and GM-CSF. In some embodiments, the agent is a mutein selected from the group consisting of IL-3 muteins, G-CSF muteins and GM-CSF muteins. In some embodiments, the mutein is an IL-3 mutein. In some embodiments, the agent is a soluble receptor which is capable of binding to IL-3.

### CROSS REFERENCE TO RELATED APPLICATIONS

This application is a continuation of U.S. patent application Ser. No. 18/591,478, filed on Feb. 29, 2024, which is a continuation of U.S. patent application Ser. No. 17/205,585, filed on Mar. 18, 2021, which is a continuation of U.S. patent application Ser. No. 16/580,012, filed on Sep. 24, 2019, which is a continuation of U.S. patent application Ser. No. 15/690,826, filed on Aug. 30, 2017, which is a continuation of U.S. patent application Ser. No. 15/193,784, filed Jun. 27, 2016, which is a continuation of U.S. patent application Ser. No. 14/517,254, filed Oct. 17, 2014, which is a continuation of U.S. patent application Ser. No. 13/499,436, filed Jun. 8, 2012, which is a 371 of International Application No. PCT/AU2010/001295, filed Oct. 1, 2010, which claims the benefit of priority of U.S. Provisional Application No. 61/247,669, filed Oct. 1, 2009, the entire contents of which are incorporated herein by reference.

### FIELD OF THE INVENTION

This invention relates to a method for the treatment of Philadelphia chromosome positive (Ph+) leukemia including chronic myeloid leukemia, and in particular it relates to a combination therapy for the treatment of this myeloproliferative disorder.

### BACKGROUND OF THE INVENTION

Many leukemia types including chronic myelogenous leukemia (CML) and acute lymphoblastic leukemia (ALL) can exhibit a chromosomal abnormality referred to as the Philadelphia chromosome (Ph). This abnormality is generated by the specific reciprocal translocation t(9; 22) (q34; q11), which fuses the Abelson kinase gene (ABL) from chromosome 9 with the breakpoint cluster region (BCR) gene of chromosome 22, leading to the BCR-ABL protein product: a constitutively active tyrosine kinase 2. BCR-ABL promotes cell survival and proliferation through several intracellular signal transduction pathways, and is responsible for malignant transformation in the disease (see Savona M and Talpaz M. Nature Reviews Cancer, May 2008).

Knowledge of the molecular structure of the BCR-ABL tyrosine kinase and the mechanisms of the disease led to development of tyrosine kinase inhibitors (TKIs), perhaps the most successful result yet born of translational medicine. Imatinib (as imatinib mesylate; Gleevec or Glivec; Novartis, Basel, Switzerland) was the first TKI to be used effectively in patients with CML. With its introduction into clinical medicine in the late 1990s, the natural history of CML has been altered very favourably for many patients. Moreover, with TKI therapy the collateral effects on normal cells are minimized owing to the relative precision of the targeted therapy. Even with this success, however, imatinib therapy is frequently not curative, acting to suppress, but not eliminate, the disease.

Furthermore, not all patients benefit from imatinib owing to resistance or intolerance. As a result, two further TKIs, dasatinib (Sprycel; Bristol-Myers Squibb) and nilotinib (Tasigna; Novartis) have been developed. Dasatinib is an inhibitor of BCR-ABL with 325 times the in vitro potency of imatinib and also inhibits Src family kinases. Nilotinib is an analogue of imatinib with enhanced specificity for BCR-ABL. However, the position of imatinib as the TKI of choice for first-line CML therapy is established at this time as this drug produces few significant side effects. The issue of side effects is important because patients with CML generally need to remain on imatinib long term as it does not always cure CML, but rather only stabilises the disorder when taken on a continuous (for example, daily) basis.

It is believed that the reason why imatinib and other TKIs are a chronic or long-term therapy is that although TKIs are effective at debulking the blasts, the leukemic stem cells are less sensitive to these TKIs, and it has been suggested that this reflects a decreased requirement of these stem cells on the underlying mutant BCR-ABL. The primitive Ph+ leukemic stem cells, which appear to be refractory to TKI therapy with imatinib, provide sanctuary for the BCR-ABL mutation and, in the absence of TKIs, pass the mutation to their progeny, which then maintain the disease. As the molecular biology of the Philadelphia chromosome and the consequent intracellular dysregulation of leukemia has been elucidated, the qualities of “stemness” have been recognized in progeny leukemia cells.

Accordingly there remains a need to develop a therapeutic regimen which alleviates the limitations of TKIs, such as the need for chronic use and the development of TKI resistance and TKI intolerance.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

### SUMMARY OF THE INVENTION

In one aspect, the present invention provides a method for the treatment of Ph+ leukemia in a patient, said method comprising administering to the patient (i) a BCR-ABL tyrosine kinase inhibitor, and (ii) an agent which selectively binds to a cell surface receptor expressed on Ph+ leukemic stem cells.

In another aspect, the present invention provides the use of (i) a BCR-ABL tyrosine kinase inhibitor, and (ii) an agent which selectively binds to a cell surface receptor expressed on Ph+ leukemia stem cells in the treatment of Ph+ leukemia in a patient or in the manufacture of a medicament.

The present invention also provides an agent for the treatment of Ph+ leukemia in a patient, which comprises (i) a BCR-ABL tyrosine kinase inhibitor, and (ii) an agent which selectively binds to a cell surface receptor expressed by Ph+ leukemic stem cells.

In yet another aspect, the invention also provides a kit which comprises (i) a BCR-ABL tyrosine kinase inhibitor, and (ii) an agent which selectively binds to a cell surface receptor expressed on Ph+ leukemic stem cells; and optionally (iii) instructions to administer said tyrosine kinase inhibitor and said agent in accordance with a method for the treatment of Ph+ leukemia in a patient.

### BRIEF DESCRIPTION OF THE DRAWINGS

FIG. 1 is a diagrammatical representation of experiments using CML samples from 3 patients looking at the cellular levels of p-STAT5 in a variety of cell culture conditions The CD123 antibody used in these experiments is 7G3 (murine antibody to human IL-3Rα).

FIG. 2 is a graphical representation of the data presented in FIG. 1 (note that to aid presentation, the data relating to the following group were not shown in the flow cytometry presented in FIG. 1: IL-3 plus BM4).

FIG. 3 shows similar experiments to those described in FIG. 1 based on 4 patients. The antibody used in these experiments is CSL362, a humanized version of 7G3 with enhanced Fc effector function.

FIG. 4 is a graphical representation of the data presented in FIG. 3 (note that to aid presentation, data relating to the following groups were not shown in the flow cytometry presented in FIG. 3: IL-3 plus BM4; IL-3 plus CSL362; and IL-3 plus dasatinib plus BM4).

FIG. 5 is a graphical representation combining the data from FIGS. 2 and 4.

FIG. 6 is a diagrammatical representation showing the effects of blocking the β-common receptor (CD131) using the antibody designated BION-1 on levels of pSTAT5 in the KU812 cell line stimulated with GM-CSF. KU812 is a myeloid precursor cell line established from the peripheral blood of a patient in blast crisis of CML.

### DETAILED DESCRIPTION OF THE INVENTION

The present invention is based on the realisation that a subset of Ph+ leukemic stem cells are able to survive by means additional to tyrosine kinase (TK) activation through BCR-ABL. As a result, this subset of stem cells are inhibited but not killed by BCR-ABL inhibitors such as imatinib.

In addition, the development of resistance to imatinib and other TKIs is an increasing clinical problem in the treatment of Ph+ leukemias, particularly CML, where long term continuous administration of the TKI is generally necessary in order to stabilise the disorder (see U.S. Pat. No. 7,799,788 to Bhalla et al).

This has led to the development in accordance with the present invention of a therapeutic regimen that combines the direct anti-leukemic effects of imatinib with an agent that can eradicate the Ph+ leukemic stem cells. This approach targets the so-called “reservoir of aberrant cells” which remains during treatment of Ph+ leukemia using TKIs, and which cause the disorder to reappear if TKI treatment is stopped.

Thus, in work leading to the present invention, a combination therapy has been developed involving the use of an agent which selectively binds to a cell surface receptor expressed on Ph+ leukemic stem cells, in particular by binding to a receptor involved in signalling by at least one of interleukin-3 (IL-3), granulocyte colony stimulating factor (G-CSF) and/or granulocyte macrophage colony stimulating factor (GM-CSF) in Ph+ leukemic stem cells, in combination with a TKI, in the treatment of Ph+ leukemia.

Interleukin-3 (IL-3) is a cytokine that stimulates production of haematopoietic cells from multiple lineages and also has an important role in host defence against certain parasitic infections. IL-3 stimulates the differentiation of multipotent hematopoietic stem cells (pluripotent) into myeloid progenitor cells as well as stimulating proliferation of cells in the myeloid lineage including, eosinophils, monocytes, basophils and B-cells. IL-3 is primarily produced and secreted by activated T lymphocytes, in response to immunological stimuli.

IL-3 exerts its activity through binding to a specific cell surface receptor known as the interleukin-3 receptor (IL-3R). IL-3R is a heterodimeric structure composed of a 70 kDa IL-3Rα (CD123) and a 120-140 kDa common □-chain (can also be referred to as IL-3R□□ or CD131). The IL-3Rα chain has a very short intracellular domain while the common β-chain has a very large cytoplasmic domain. IL-3Rα binds IL-3 with relatively low affinity. In the presence of common β-chain, however, IL-3Rα has a much higher affinity for IL-3. It is not clear how signal transduction occurs following IL-3 binding, however recent studies suggest signalling requires formation of a higher order complex comprising a dodecamer. The common β-chain is also shared by the receptors for IL-5 and GM-CSF. Cells known to express IL-3 receptor include normal hematopoietic progenitors as well as more mature cells of various hematopoietic lineages including monocytes, macrophages, basophils, mast cells, eosinophils, and CD5⁺ B cell sub-populations. Non-hematopoietic cells have also been shown to express the receptor including some endothelial cells, stromal cells, dendritic cells and Leydig cells.

Granulocyte colony-stimulating factor (G-CSF) stimulates the proliferation and differentiation of neutrophil precursors via interaction with a specific cell surface receptor, the G-CSF receptor (G-CSF-R) (CD114). The G-CSF-R has been cloned and is functionally active in several different cells types. The G-CSF-R is believed to consist of a single chain that is activated through ligand induced homodimersation as has been shown for the erythropoietin and growth hormone receptors (EPO-R, GH-R). The G-CSF-R does not contain an intrinsic protein kinase domain, although tyrosine kinase activity seems to be required for transduction of the G-CSF signal.

Granulocyte-macrophage colony-stimulating factor (GM-CSF) is a growth and differentiation factor for a variety of haemopoietic progenitor cells (including those for neutrophils, macrophages, eosinophils, megakaryocytes and erythroid cells) and can also functionally activate mature neutrophils, eosinophils and macrophages. All of the actions of GM-CSF are thought to be mediated through the interaction of GM-CSF with specific cell surface receptors. These receptors consist of a specific alpha-chain, GM-CSFRα (CD116), which binds GM-CSF with low affinity, and a common beta-chain (CD131), which by itself does not bind GM-CSF with detectable affinity, and is shared by the alpha-chains for the interleukin-3 and interleukin-5 receptors (as noted above). The alpha-beta complex generates high-affinity binding sites for GM-CSF, and is required for cell signalling.

An advantage of the combination therapy of the present invention is that it addresses the problems associated with the use of TKI monotherapy, such as the resistance problem, by also providing treatment of the patient with an agent (such as a monoclonal antibody) which selectively binds to a cell surface receptor expressed on Ph+ leukemic stem cells. A further advantage is that many patients cannot tolerate long-term treatment with TKIs or antibodies such as those used in the combination therapy of the present invention, and this combination therapy approach may reduce the time during which the TKIs and the antibodies are administered to the patient.

In one aspect, the present invention provides a method for the treatment of Ph+ leukemia in a patient, said method comprising administering to the patient (i) a BCR-ABL tyrosine kinase inhibitor, and (ii) an agent which selectively binds to a cell surface receptor expressed on Ph+ leukemic stem cells.

The patient may be human.

The Ph+ leukemia may be selected from chronic myeloid leukemia (CML), acute lymphoid leukemia (ALL) and acute myeloid leukemia (AML). More particularly, the Ph+ leukemia may be chronic myeloid leukemia (CML).

Reference herein to “treatment” is to be considered in its broadest context. The term “treatment” does not necessarily imply that a patient is treated until recovery. Accordingly, treatment includes reduction or amelioration of the symptoms of Ph+ leukemia in the patient, as well as halting or at least retarding progress of, reducing the severity of, or eliminating Ph+ leukemia.

The treatment regime of the present invention is a combination therapy for Ph+ leukemia. Thus, administration of the TKI to the patient will usually be a continuous, for example daily, therapy, and administration to the patient of the agent which selectively binds to a cell surface receptor expressed on Ph+ leukemic stem cells may be carried out simultaneously with administration of the TKI, for example daily, every two or three days, or weekly or even less frequently. In an alternative combination therapy regimen, the TKI is administered to the patient until the patient is considered to have reached the clinical remission stage, and the disorder has been stabilised, and then the agent which binds to a cell surface receptor expressed on Ph+ leukemic stem cells is added to the therapeutic regimen. In this regard, a CML patient can be considered to be in “clinical remission” if there are less than 5% blasts in a bone marrow sample taken from the patient.

In this embodiment the present invention provides the use of other TKIs with specificity to BCR-ABL such as dasatinib, nilotinib, bosutinib, axitinib, cediranib, crizotinib, damnacanthal, gefitinib, lapatinib, lestaurtinib, neratinib, semaxanib, sunitinib, toceranib, tyrphostins, vandetanib, vatalanib, INNO-406, AP24534 (Ariad Pharmaceuticals), XL228 (Exelixis), PHA-739358 (Nerviano), MK-0457, SGX393 and DC-2036. Effective oral regimens for TKIs such as imatinib or nilotinib in treatment of Ph+ leukemia such as CML have been found to be doses of 400 mg per day, with high-dose regimens consisting of 600 or 800 mg per day. In one embodiment, the TKI is imatinib. In another embodiment, the TKI is not imatinib. In a related aspect, the TKI is nilotinib. In a further aspect, the TKI is dasatinib.

The method of the present invention includes administration of an agent which selectively binds to a cell surface receptor expressed on Ph+ leukemic stem cells, and in one embodiment is an agent capable of binding to a receptor involved in signalling by at least one of IL-3, G-CSF and GM-CSF. The agent may be one which binds to a receptor involved in IL-3 signalling; however the method also encompasses administration of other agents which bind to receptors involved in G-CSF and/or GM-CSF signalling, either alone or in combination. Accordingly, the combination therapy which is administered to the patient may comprise a single agent which binds to a receptor involved in IL-3, G-CSF or GM-CSF signalling, or alternatively it may comprise a combination of such agents. In some embodiments the patient's Ph+ leukemic stem cells may be sampled and tested for responsiveness to IL-3, G-CSF or GM-CSF in order to select the appropriate agent/s to treat the patient.

As used herein, the term “agent which selectively binds to a cell surface receptor expressed on Ph+ leukemic stem cells” refers to an agent which is capable of binding to an appropriate cell surface receptor, such as an IL-3, G-CSF and/or GM-CSF receptor, and which will selectively facilitate Ph+ leukemic stem cell death without leading to collateral damage or side-effects which would be unacceptable to a patient during treatment.

In an embodiment of combination therapy of the present invention, the agent which selectively binds to a cell surface receptor expressed by Ph+ leukemic stem cells is believed to enhance the efficacy of the TKI by either blocking or inhibiting IL-3, G-CSF and/or GM-CSF signalling events in the stem cells or by directly eliminating “resistant” stem cells by Fc effector or cytotoxic activity, or any combination thereof, or by any other mechanism.

In one aspect, the agent is an antigen binding molecule which selectively binds to a receptor selected from the group consisting of IL-3Rα, G-CSFR, GM-CSFRα, and the beta-common receptor for IL-3 and GM-CSF.

As used herein, the term “antigen binding molecule” refers to an intact immunoglobulin, including monoclonal antibodies, such as bispecific, chimeric, humanized or human monoclonal antibodies, or to antigen-binding (including, for example, Fv, Fab, Fab′ and F(ab′)₂ fragments) and/or variable-domain-comprising fragments of an immunoglobulin that compete with the intact immunoglobulin for specific binding to the binding partner of the immunoglobulin, e.g. a host cell protein. Regardless of structure, the antigen-binding fragments bind with the same antigen that is recognized by the intact immunoglobulin. Antigen-binding fragments may be produced synthetically or by enzymatic or chemical cleavage of intact immunoglobulins or they may be genetically engineered by recombinant DNA techniques. The methods of production of antigen binding molecules and fragments thereof are well known in the art and are described, for example, in Antibodies, A Laboratory Manual, Edited by E. Harlow and D. Lane (1988), Cold Spring Harbor Laboratory, Cold Spring Harbor, New York, which is incorporated herein by reference.

In one embodiment, the antigen binding molecule is a monoclonal antibody.

In this embodiment of the invention, the antigen binding molecule may comprise an Fc region or a modified Fc region, more particularly a Fc region which has been modified to provide enhanced effector functions, such as enhanced binding affinity to Fc receptors, antibody-dependent cell-mediated cytotoxicity (ADCC), antibody-dependent cell-mediated phagocytosis (ADCP) and complement-dependent cytotoxicity (CDC). For the IgG class of antibodies, these effector functions are governed by engagement of the Fc region with a family of receptors referred to as the Fcγ receptors (FcγRs) which are expressed on a variety of immune cells. Formation of the Fc/FcγR complex recruits these cells to sites of bound antigen, typically resulting in signalling and subsequent immune responses. Methods for optimizing the binding affinity of the FcγRs to the antibody Fc region in order to enhance the effector functions, in particular to alter the ADCC and/or CDC activity relative to the “parent” Fc region, are well known to persons skilled in the art. These methods can include modification of the Fc region of the antibody to enhance its interaction with relevant Fc receptors and increase its potential to facilitate ADCC and ADCP. Enhancements in ADCC activity have also been described following the modification of the oligosaccharide covalently attached to IgG1 antibodies at the conserved Asn²⁹⁷ in the Fc region.

The term “selectively binds”, as used herein, in reference to the interaction of an antigen binding molecule, e.g. an antibody or antibody fragment, and its binding partner, e.g. an antigen, means that the interaction is dependent upon the presence of a particular structure, e.g. an antigenic determinant or epitope, on the binding partner. In other words, the antibody or antibody fragment preferentially binds or recognizes the binding partner even when the binding partner is present in a mixture of other molecules or organisms.

In another embodiment of the present invention, the agent may be a mutein selected from the group consisting of IL-3 muteins, G-CSF muteins and GM-CSF muteins, wherein the mutein selectively binds to a receptor selected from the group consisting of IL-3R, G-CSFR, GM-CSFR but does not lead to signal activation or at least results in reduced cytokine-induced signal activation. In an embodiment, the mutein is an IL-3 mutein which binds to IL-3R but either does not lead to or at least results in reduced IL-3 signal activation. Generally, these ‘IL-3 muteins’ include natural or artificial mutants differing by the addition, deletion and/or substitution of one or more contiguous or non-contiguous amino acid residues. An example of an IL-3 mutein which binds to IL-3R but exhibits reduced IL-3 signal activation is a 16/84 C→A mutant. IL-3 muteins may also include modified polypeptides in which one or more residues are modified to, for example, increase their in vivo half life. This could also be achieved by attaching other elements such as a PEG group. Methods for the PEGylation of polypeptides are well known in the art.

In another embodiment of the present invention, the agent may be a soluble receptor which is capable of binding to IL-3. Examples of such soluble receptors include the extracellular portion of IL-3Rα or a fusion protein comprising the extracellular portion of IL-3Rα fused to the extracellular portion of common β-chain.

The agent which is capable of binding to a receptor involved in signalling by at least one of IL-3, G-CSF and GM-CSF is administered in an effective amount. An “effective amount” means an amount necessary at least partly to attain the desired response or to delay or inhibit progression or halt altogether, the progression of the particular condition being treated. The amount varies depending upon the health and physical condition of the individual to be treated, the racial background of the individual to be treated, the degree of protection desired, the formulation of the composition, the assessment of the medical situation, and other relevant factors. It is expected that the amount will fall in a relatively broad range that can be determined through routine trials. If necessary, the administration of the agent may be repeated one or several times. The actual amount administered will be determined both by the nature of the condition which is being treated and by the rate at which the agent is being administered.

For certain applications, it is envisioned that pharmacologic compounds will be useful when attached to the agent, particularly cytotoxic or otherwise anti-cellular agents having the ability to kill or suppress the growth or cell division of Ph+ leukemic stem cells. In general, the invention contemplates the use of any pharmacologic compound that can be conjugated to an agent and delivered in active form to the targeted cell. Exemplary anti-cellular compounds include chemotherapeutic compounds, radioisotopes as well as cytotoxins. In the case of chemotherapeutic compounds, compounds such as a hormone such as a steroid; an antimetabolite such as cytosine arabinoside, fluorouracil, methotrexate or aminopterin; an anthracycline; mitomycin C; a vinca alkaloid; demecolcine; etoposide; mithramycin; macrolide antibiotics such as maytansines; enediyne antibiotics such as calicheamicins, CC-1065 and derivatives thereof, or an alkylating agent such as chlorambucil or melphalan, will be particularly preferred. Other embodiments may include compounds such as a coagulant, a cytokine, growth factor, bacterial endotoxin or the lipid A moiety of bacterial endotoxin. In any event, it is proposed that compounds such as these may be successfully conjugated to the agent in a manner that will allow their targeting, internalization, release or presentation to blood components at the site of the targeted Ph+ leukemic stem cells as required using known conjugation technology.

In certain embodiments, cytotoxic compounds for therapeutic application are conjugated to an antibody recognising either IL-3Rα, G-CSFR, GM-CSFRα or the beta-common receptor for IL-3 and GM-CSF. The cytotoxic compounds for therapeutic application will include generally a plant-, fungus- or bacteria-derived toxin, such as an A chain toxin, a ribosome inactivating protein, a-sarcin, auristatin, aspergillin, restirictocin, a ribonuclease, diphtheria toxin or Pseudomonas exotoxin, to mention just a few examples. The use of toxin-antibody constructs is well known in the art of immunotoxins, as is their attachment to antibodies. Of these, a particularly preferred toxin for attachment to antibodies will be a deglycosylated ricin A chain. Deglycosylated ricin A chain is preferred because of its extreme potency, longer half-life, and because it is economically feasible to manufacture at clinical grade and scale.

In other embodiments, the cytotoxic compound may be a radioisotope. Radioisotopes include α-emitters such as, for example, 211Astatine, 212Bismuth and 213Bismuth, as well as β-emitters such as, for example, 131Iodine, 90Yttrium, 177Lutetium, 153Samarium and 109Palladium, and Auger emitters such as, for example, 111Indium.

In accordance with the present invention, the agent may be administered to a patient by a parenteral route of administration. Parenteral administration includes any route of administration that is not through the alimentary canal (that is, not enteral), including administration by injection, infusion and the like. Administration by injection includes, by way of example, into a vein (intravenous), an artery (intraarterial), a muscle (intramuscular) and under the skin (subcutaneous). The agent may also be administered in a depot or slow release formulation, for example, subcutaneously, intradermally or intramuscularly, in a dosage which is sufficient to obtain the desired pharmacological effect.

In one particular embodiment, the agent which binds to a receptor involved in IL-3 signalling is an antigen binding molecule, more particularly a monoclonal antibody, which binds selectively to IL-3Rα (CD123). Thus, the agent may be monoclonal antibody (mAb) 7G3, raised against CD123, which has previously been shown to inhibit IL-3-mediated proliferation and activation of both leukemic cell lines and primary cells (see U.S. Pat. No. 6,177,078 to Lopez). Alternatively, the agent may be the monoclonal antibody CSL360, a chimeric antibody obtained by grafting the light variable and heavy variable regions of the mouse monoclonal antibody 7G3 onto a human IgG1 constant region. Like 7G3, CSL360 binds to CD123 (human IL-3Rα) with high affinity, competes with IL-3 for binding to the receptor and blocks its biological activities. The mostly human chimeric antibody CSL360, can thus potentially also be used to target and eliminate leukemic stem cells. CSL360 also has the advantage of potential utility as a human therapeutic agent by virtue of its human IgG1 Fc region which would be able to initiate some level of effector activity in a human setting. Moreover, it is likely that in humans it would show reduced clearance relative to the mouse 7G3 equivalent and be less likely to be immunogenic. Further examples of this agent include humanised antibody variants of 7G3, such as CSL362 (which also has enhanced Fc effector function), fully human anti-CD123 antibodies and anti-CD123 antibodies with enhanced effector function such as ADCC activity, examples of which are disclosed in International Patent Application No. PCT/AU2008/001797 (WO 2009/070844).

The agent which binds to a receptor involved in G-CSF signalling may be, for example, an antibody recognising G-CSFR disclosed in WO 95/21864. Similarly, the agent which binds to a receptor involved in GM-CSF signalling may be, for example, an antibody recognising GM-CSFRα disclosed in International Patent Application No. PCT/AU93/00516 (WO 94/09149) or International Patent Application No. PCT/GB2007/001108 (WO 2007/110631). In yet another embodiment, the agent may be an antibody recognising the beta-common receptor for IL-3 and GM-CSF, for example, as disclosed in International Patent Application No. PCT/AU97/00049 (WO 97/28190).

In another aspect, the present invention provides the use of (i) a BCR-ABL tyrosine kinase inhibitor, and (ii) an agent which selectively binds to a cell surface receptor expressed on Ph+ leukemic stem cells, in, or in the manufacture of a medicament for, the treatment of Ph+ leukemia in a patient.

In yet another aspect, the present invention provides a composition for the treatment of Ph+ leukemia in a patient, which comprises (i) a BCR-ABL tyrosine kinase inhibitor, and (ii) an agent which selectively binds to a cell surface receptor expressed on Ph+ leukemic stem cells.

The invention also provides a kit which comprises (i) a BCR-ABL tyrosine kinase inhibitor, and (ii) an agent which selectively binds to a cell surface receptor expressed on Ph+ leukemic stem cells; and optionally (iii) instructions to administer said tyrosine kinase inhibitor and said agent in accordance with a method for the treatment of Ph+ leukemia in a patient.

Each of the components of this kit may be supplied in a separate container, and the instructions, if present, may direct the administration of the components of the kit at different times and in different dosage forms from one another.

The phrase “combination therapy” (or “co-therapy”) as used herein embraces the administration of a BCR-ABL tyrosine kinase inhibitor and an agent which selectively facilitates Ph+ leukemic stem cell death by binding to a cell surface receptor expressed on the leukemic stem cell as part of a specific treatment regimen intended to provide a beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks, or even months depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule or intravenous injection having a fixed ratio of each therapeutic agent or in multiple, single capsules or intravenous injections for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered orally while the other therapeutic agents of the combination may be administered by intravenous injection. Alternatively, for example, all therapeutic agents may be administered orally or all therapeutic agents may be administered by intravenous injection. In one example of sequential administration, the BCR-ABL tyrosine kinase inhibitor is administered first to stabilise the disorder (i.e. wherein the patient has less than 5% blasts in the bone marrow). Once the disorder has been stabilised, the agent which selectively facilitates Ph+ leukemic stem cell death by binding to a cell surface receptor expressed on the leukemic stem cell is added to the therapeutic regimen.

In the combination therapy of the present invention, the BCR-ABL TKI may be administered to the patient in oral dosage form, while the agent which selectively binds to a cell surface receptor expressed on Ph+ leukemia stem cells may be administered parenterally.

Compositions suitable for oral administration may be presented as discrete units such as tablets, capsules, cachets, caplets or lozenges, each containing a predetermined amount or dosage of the active component, or as a solution or suspension in an aqueous or non-aqueous carrier liquid such as a syrup, an elixir, or an emulsion.

Compositions suitable for parenteral administration conveniently comprise a sterile aqueous preparation of the active component which is preferably isotonic with the blood of the recipient. This aqueous preparation may be formulated according to known methods using suitable dispersing or wetting agents and suspending agents. The sterile injectable preparation may also be a sterile injectable solution or suspension in a non-toxic parenterally-acceptable diluent or solvent, for example as a solution in polyethylene glycol and lactic acid. Among the acceptable vehicles and solvents that may be employed are water, Ringer's solution, suitable carbohydrates (e.g. sucrose, maltose, trehalose, glucose) and isotonic sodium chloride solution. In addition, sterile, fixed oils are conveniently employed as a solvent or suspending medium. For this purpose, any bland fixed oil may be employed including synthetic mono- or di-glycerides. In addition, fatty acids such as oleic acid find use in the preparation of injectables.

The formulation of such therapeutic compositions is well known to persons skilled in this field. Suitable pharmaceutically acceptable carriers and/or diluents include any and all conventional solvents, dispersion media, fillers, solid carriers, aqueous solutions, coatings, antibacterial and antifungal agents, isotonic and absorption delaying agents, and the like. The use of such media and agents for pharmaceutically active substances is well known in the art, and it is described, by way of example, in Remington's Pharmaceutical Sciences, 18th Edition, Mack Publishing Company, Pennsylvania, USA. Except insofar as any conventional media or agent is incompatible with the active ingredient, use thereof in the pharmaceutical compositions of the present invention is contemplated. Supplementary active ingredients can also be incorporated into the compositions.

The present invention is further illustrated by the following non-limiting Example(s).

### Example 1—Materials and Methods

### CD34+ Cells

Mononuclear cells from blood collected from newly diagnosed CML-CP patients and normal donors were isolated by density gradient centrifugation using Lymphoprep (Axis-Shield, Norway). CD34-positive cells were further purified by magnetic-assisted cell sorting using CD34 mAb-coupled magnetic micro-beads (Miltenyi Biotech, Germany).

pSTAT5 Assay (Used to Measure Cytokine Induced Signalling)

CD34+ progenitor cells were cultured in serum-deprived media (SDM, containing IMDM, 2 mM L-glutamine, 1% BSA, 1 U/ml insulin, 0.2 mg/ml transferrin, 0.1 mM 2-mercaptoethanol and 20 □g/ml low-density lipoproteins). For examining STAT5 phosphorylation levels, cells were pre-treated with CSL362 or 7G3 (0.1 □g/ml) and/or Dasatinib (100 nM; Symansis, New Zealand) as indicated prior to stimulation with 20 ng/ml IL-3 (Pepro Tech, USA). Following PFA-fixation and permeabilisation with ice-cold methanol, cells were stained with Alexa⁴⁸⁸-conjugated pY694-STAT5 antibody or the appropriate isotype control (Phosflow, BD Biosciences, USA) and analysed by flow cytometry using a FC500 Terpsichore (Beckman Coulter, USA).

### Data Analysis

Flow cytometry data was analysed using FCS Express V3 (DeNovo Software, USA).

GraphPad Prism 5 (GraphPad Software, USA) was used for further data presentation and statistical analysis using two tail Student t test.

### Example 2

This example shows that mAb 7G3 and dasatinib cooperate in attenuating IL-3 induced phosphorylation in CML patient primary CD34+ cell samples.

CD34⁺ cells from newly diagnosed CML-chronic phase (CML-CP) patients (n=3) were incubated with 100 nM Dasatinib, 7G3 (100 ng/ml) as indicated and consecutively stimulated with 20 ng/ml IL-3 for 10 min prior to PFA-fixation and methanol-permeabilisation. STAT5 phosphorylation was determined by flow cytometry using a Alexa488-conjugated pY694-STAT5 antibody (Phosflow, BD). (Baseline: represents the basal level of STAT5 phosphorylation in unstimulated cells); IL-3: p-STAT5 expression in cells cultured with 20 ng/ml IL-3; IL-3+Das 100 nM: p-STAT5 expression in cells cultured with 100 nM dasatinib and 20 ng/ml IL-3; IL-3+Das 100 nM+7G3: p-STAT5 expression in cells cultured with 100 nM dasatinib, 20 ng/ml IL-3 and 7G3 (110 ng/ml).

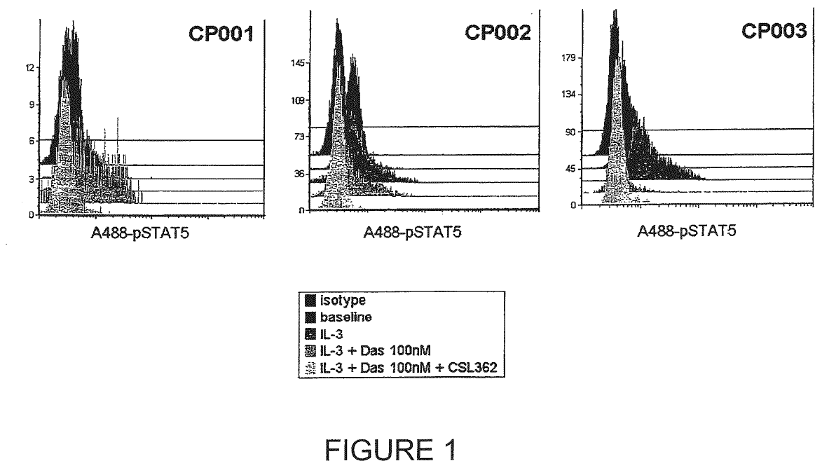

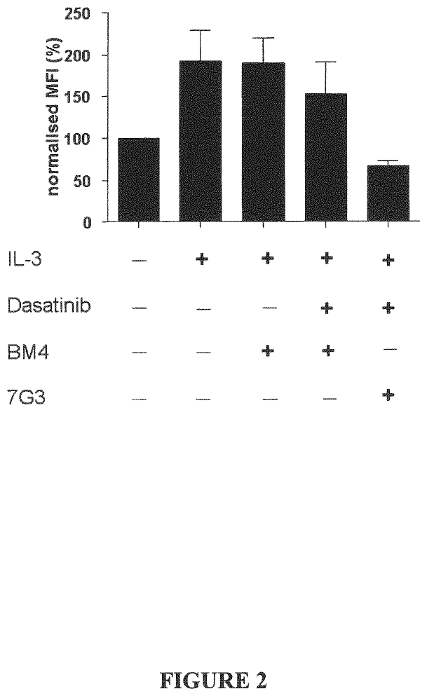

FIG. 1 and FIG. 2 (a graphical representation of the data presented in FIG. 1) show that in the presence of IL-3, dasatinib alone can only partially inhibit IL-3-induced STAT5 phosphorylation, however the combination of dasatinib and mAb 7G3 can inhibit IL-3-induced STAT5 phosphorylation completely (n=3).

### Example 3

This example shows that mAb CSL362 (a humanized and Fc effector enhanced variant of 7G3) and dasatinib cooperate in attenuating IL-3-induced STAT5 phosphorylation in CML patient primary CD34+ cell samples.

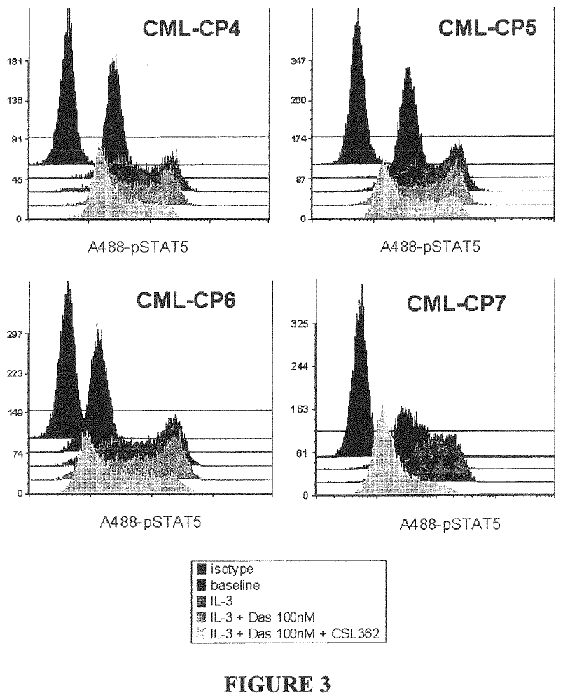

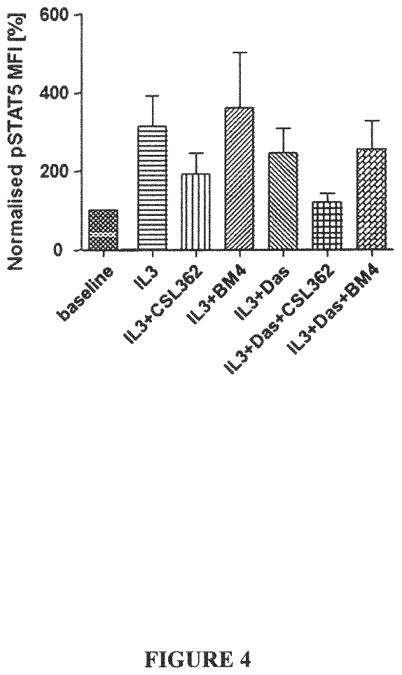

Freshly thawed CD34+ cells from newly diagnosed chronic phase CML patients after 1 h of recovery in SDM were incubated with 100 nM Dasatinib, 0.1 □g/ml CSL362 or BM4 (an isotype-matched control for CSL362) or CSL362 and Dasatinib as indicated and consecutively stimulated with 20 ng/ml IL-3 for 10 min prior to PFA-fixation and methanol-permeabilisation. STAT5 phosphorylation was determined by flow cytometry using a Alexa488-conjugated pY694-STAT5 antibody (Phosflow, BD). A488-pSTAT fluorescence histograms of individual patient samples are shown in FIG. 3 (cells only represents the unstimulated level of STAT5 phosphorylation in these cells). FIG. 4 is a graphical representation of the data presented in FIG. 3. Mean fluorescence intensity normalised to the baseline STAT5 phosphorylation +/− SEM is plotted (n=4).

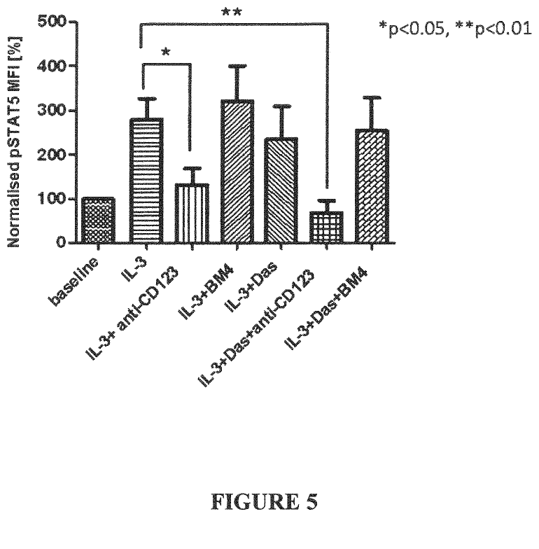

FIG. 5 is a graphical representation combining the data from FIGS. 2 and 4 (n=7) that shows all CML patient data including both mAbs 7G3 and CSL362. These data demonstrate that the combination of an antibody (cither 7G3 or CSL362) with dasatinib is more effective than dasatinib or antibody used alone.

### Example 4

This example shows that BION-1, an anti-β-common receptor (CD131) antibody cooperates with dasatanib to inhibit GM-CSF signalling in KU812 CML cells. KU812 cells (a myeloid precursor cell line established from the peripheral blood of a patient in blast crisis of CML) were cultured with 100 nM dasatinib with or without Bion-1 (100 nM) and constitutively stimulated with GM-CSF (4 ng/ml) for 10 minutes. STAT5 phosphorylation was determined by flow cytometry using a Alexa488-conjugated pY694-STAT5 antibody (Phosflow, BD). (Baseline: represent the basal STAT5 phosphorylation in unstimulated cells; GM-CSF: p-STAT5 expression in cells cultured with GM-CSF; GM-CSF+Das 100 nM: p-STAT5 expression in cells cultured with GM-CSF and dasatinib 100 nM; GM-CSF+Das 100 nM+ Bion-1: p-STAT5 expression in cells cultured with 100 nM dasatinib, GM-CSF and Bion-1.

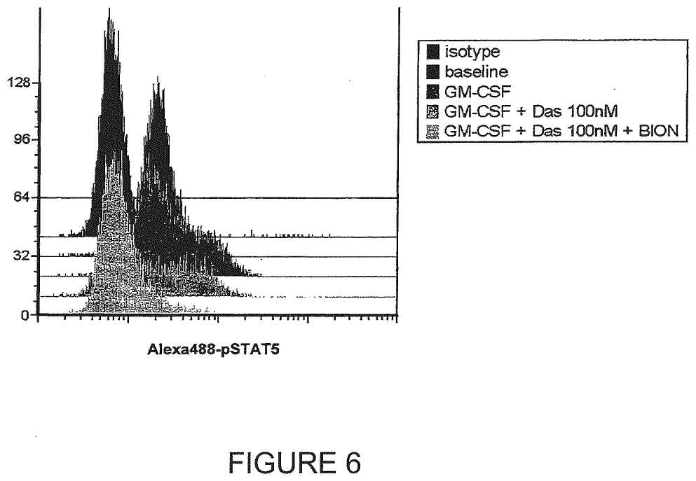

FIG. 6 shows that in KU812 cells, dasatinib alone does not inhibit p-STAT5 phosphorylation in the presence of GM-CSF, however the combination of dasatinib and BION-1 inhibits GM-CSF-induced p-STAT5 phosphorylation.

### Example 5

This Example describes a randomized multi-center study comparing the effect on malignant stem cells of treatment with anti-CD123 monoclonal antibody (mAb) (or mAb to GM-CSF or G-CSF) plus imatinib (or other TKI) or treatment with imatinib alone in newly diagnosed chronic phase (CP) chronic myeloid leukemia (CML) patients.

Estimated Number of Study Centers and Countries/Regions: Appr. 6-8 sites in Australia and the USA. Stem cell analyses are performed in the USA (e.g. at University of Washington in Seattle or at Johns Hopkins Kimmel Cancer Center in Baltimore) and in Australia (CSL/University of Melbourne).

### Study Phase: II

The research hypothesis is that treatment with anti-CD123 monoclonal antibody (mAb) (an example of which has previously been demonstrated to be safe in a phase I trial) plus imatinib results in more effective and more rapid depletion of the Philadelphia chromosome (Ph)—positive stem cell pool within 6 months of therapy than imatinib alone in newly diagnosed chronic phase (CP) chronic myeloid leukemia (CML) patients. The study duration is 18 months and approximately 60 patients are recruited to the study.

Primary Objective: To compare the number of Ph-positive cells in the stem cell compartment in newly diagnosed CP CML patients treated with anti-CD123 mAb plus imatinib vs. imatinib alone.

Study Design: This study is an open-label randomized Phase II trial in newly diagnosed CML patients in CP. Patients are randomized to receive anti-CD123 mAb at 3 mg/kg by intravenous infusion (infusions may be made weekly, fortnightly, or monthly) plus continuous oral imatinib at a starting dose of 400 mg once daily, or single agent oral imatinib at a starting dose of 400 mg once daily.

Duration of Study: The study is open for enrollment until the planned number of 60 patients is randomized. All patients are treated and/or followed for up to 18 months. Number of Patients per Group: In the randomized, two-arm comparative study, approximately 40 patients are randomized, 20 patients to combination therapy and 20 to monotherapy imatinib. Additional patients are recruited in the event that insufficient numbers of representative samples have been obtained from the first 40 patients, including bone marrow samples from patients treated for at least 12 weeks.

Study Population: Patients 18 years or older with a newly diagnosed CP CML, not previously treated with any systemic treatments for CML.

Study Assessments and Endpoints: The safety of combination therapy of leukemia patients with anti-CD123 mAb plus imatinib is determined in a prior Phase I study. The dose of anti-CD123 mAb selected for use in combination with imatinib is determined in that Phase 1 study.

Safety in this Phase II study is assessed using the NCI-CTC version 3. There are comparative analyses of the incidences of all adverse events, toxicities and laboratory abnormalities in the two treatment arms.

Efficacy is assessed by stem cell assays of patient's bone marrow samples. All stem cell assays are based on the pre-selection of CD34+ cells from bone marrow (BM) aspirates using paramagnetic beads. The CD34+ fraction is further subdivided based on the expression of CD38 marker (positive vs. negative) using a sorting flow cytometer. Multiparameter flow cytometric immunophenotyping is used to identify the cellular fractions containing the leukemic stem cell population.

The primary endpoint is a comparison of proportion of Ph-positive cells in stem cell compartments (CD34+CD38neg and CD34+CD38+) at 6 months between the study arms. Secondary endpoints are comparisons between treatment arms for:
(1) the number of Ph-positive cells in all stem cell compartments at 1 and 3 months,
(2) BCR-ABL mRNA transcript levels measured using RQ-PCR (real time quantitative PCR) on blood samples taken at 1, 3, 6, 12 and 18 months, and
(3) rate of complete cytogenetic response (CCyR) within 3, 6, 12 and 18 months.

The Phase II clinical study will include an additional (3^{rd}) treatment arm of 20 patients who will be treated with imatinib monotherapy for a designated duration (e.g. 4-6 months). A bone marrow sample taken at the conclusion of the imatinib monotherapy period will enable diagnosis of patients with persistent/residual Ph-positive cells in stem cell compartments, and quantitation of the residual leukaemia cells. These patients will be treated with the combination of imatinib (continuing) plus anti-CD123 mAb for an additional period of 6 months. The primary endpoint assessed in these patients is change (reduction/elimination) in the Ph-positive cells in stem cell compartments at 3 and 6 month time points after starting combination therapy. Secondary endpoints will be
(1) attainment of complete cytogenetic response (CCyR) within 3, and 6 months of starting combination therapy;
(2) attainment of major molecular response (≥3 log reduction in BCR-ABL mRNA);
(3) attainment of complete molecular remission (negativity by RQ-PCR).

### Example 6—Effect of Combined Imatinib and Anti-Cytokine Antibody Therapy on CML Stem Cells In Vitro: Analysis of Survival of CD34⁺CD38⁻ CML Stem Cells

The CD34+CD38-cells are sorted by staining with CD34-APC and CD38-PE antibodies (Becton, Dickinson and Company) using a BD FacsARIA cell sorter.

Sorted cells are plated at 1.5×10⁵ cells/ml in IMDM/10% FCS with or without additional cytokines (IL-3, GM-CSF, G-CSF, SCF, IL-6, flt-3 ligand) and treated with the following regimes with final concentrations as shown:
1. control
2. 1-100 μg/mL anti-CD123 mAb
3. 1-100 μg/mL anti-CD131 mAb
4. 1-100 μg/mL anti-CD116 mAb
5. 1-100 μg/mL anti-CD114 mAb
6. 0.01-10 μM Imatinib or other TKI
7. 1-100 μg/mL anti-CD123 mAb+0.01-10 μM Imatinib or other TKI
8. 1-100 μg/mL anti-CD123 mAb+1-100 μg/mL anti-CD116 mAb+0.01-10 μM Imatinib or other TKI
9. 1-100 μg/mL anti-CD123 mAb+1-100 μg/mL anti-CD116 mAb+1-100 μg/mL anti-CD114 mAb+0.01-10 μM Imatinib or other TKI
10. 1-100 μg/mL anti-CD131 mAb+1-100 μg/mL anti-CD114 mAb+0.01-10 μM Imatinib or other TKI

Cells are analysed for viability at 24 h, 48 h and 72 h by flow cytometry after staining with Annexin-V-FITC and 7-AAD (BD Biosciences) as described (Jin, L et al., Cell Stem Cell 2009, 5:31-42). Absolute cell numbers are also assessed by flow cytometry by the addition of either Tru-Count beads (BD Biosciences) or Flow-Count Fluorospheres (Beckman Coulter).

### Example 7—Effect of Combined Imatinib and Anti-Cytokine Antibody Therapy on CML Cells In Vitro: Analysis of Effects on Colony Forming Cell Activity

Bulk CML tumour cells (1×10⁵) are placed into suspension cultures in IMDM supplemented with BIT (Stem Cell Technologies) with or without additional cytokines (IL-3, GM-CSF, G-CSF, SCF, IL-6, flt-3 ligand). Test conditions with final concentrations as shown include;
1. control
2. 1-100 μg/mL anti-CD123 mAb
3. 1-100 μg/mL anti-CD131 mAb
4. 1-100 μg/mL anti-CD116 mAb
5. 1-100 μg/mL anti-CD114 mAb
6. 0.01-10 μM Imatinib or other TKI
7. 1-100 μg/mL anti-CD123 mAb+0.01-10 μM Imatinib or other TKI
8. 1-100 μg/mL anti-CD123 mAb+1-100 μg/mL anti-CD116 mAb+0.01-10 μM Imatinib or other TKI
9. 1-100 μg/mL anti-CD123 mAb+1-100 μg/mL anti-CD131 mAb+1-100 μg/mL anti-CD116 mAb+1-100 μg/mL anti-CD114 mAb+0.01-10 μM Imatinib or other TKI
10. 1-100 μg/mL anti-CD131 mAb+1-100 μg/mL anti-CD114 mAb+0.01-10 μM Imatinib or other TKI

Culture supernatant is renewed twice weekly and growth monitored by trypan-blue exclusion over 2-3 weeks. At the end of the culture period, cells are placed into semi-solid methylcellulose progenitor media (Stem Cell Technologies) for 14-18 days and assessed for the formation of BCR-ABL+ colonies as described (Jiang, X et al., Leukemia 2007, 21:926-935).

### Example 8—Effect of Combined Imatinib and Anti-Cytokine Antibody Therapy on CML Stem Cells In Vitro: Analysis of Effects on Cell Proliferation

Bulk CML tumour cells (1×10⁴), sorted CD34+ or sorted CD34+CD38− are placed into suspension cultures in IMDM supplemented with BIT (Stem Cell Technologies) with or without additional cytokines (IL-3, GM-CSF, G-CSF, SCF, IL-6, flt-3 ligand). Test conditions with final concentrations as shown will include;
1. control
2. 1-100 μg/mL anti-CD123 mAb
3. 1-100 μg/mL anti-CD131 mAb
4. 1-100 μg/mL anti-CD116 mAb
5. 1-100 μg/mL anti-CD114 mAb
6. 0.01-10 μM Imatinib or other TKI
7. 1-100 μg/mL anti-CD123 mAb+0.01-10 μM Imatinib or other TKI
8. 1-100 μg/mL anti-CD123 mAb+1-100 μg/mL anti-CD116 mAb+0.01-10 μM Imatinib or other TKI
9. 1-100 μg/mL anti-CD123 mAb+1-100 μg/mL anti-CD131 mAb+1-100 μg/mL anti-CD116 mAb+1-100 μg/mL anti-CD114 mAb+0.01-10 μM Imatinib or other TKI
10. 1-100 μg/mL anti-CD131 mAb+1-100 μg/mL anti-CD114 mAb+0.01-10 μM Imatinib or other TKI

After 4 days in culture, cell proliferation is measured by viable cell counts determined by hemocytometer counts of trypan blue-excluding cells or as [³H]-thymidine incorporation as described (Jiang, X et al., Proc. Nat. Acad. Sci. 1999, 96:12804-12809). For the latter, [³H]-thymidine is added to the wells for a further 12 h. Cells are harvested onto glass-fibre filters and thymidine incorporation measured.

### Example 9—Effect of Anti-CD123 mAb on CML Stem Cells In Vitro: Analysis of Antibody-Dependent Cellular Cytotoxicity (ADCC)

Imatinib-resistant CML stem cell sensitivity to ADCC is determined. For this CD34+CD38-cells are sorted by staining with CD34-APC and CD38-PE antibodies (Becton, Dickinson and Company) using a BD FacsARIA cell sorter. Sorted cells are used as target cells in ADCC assays with purified natural killer (NK) cells from normal donors as described (Lazar et al., Engineered antibody Fc variants with enhanced effector function. Proc Natl Acad Sci USA. 2006 103(11): 4005-10). Target cells (CML cells; 1×10⁵ cells) are incubated with varying amounts of anti-CD123 antibodies (0.01-10 μg/mL) in the presence of NK cells at a ratio of (1:5; CML:NK). NK cells are purified from normal buffy packs using Miltenyi Biotec's NK Isolation Kit (Cat #130-092-657). The culture is incubated for a period of four hours at 37° C. in presence of 5% CO₂. Cell lysis is measured by the release of LDH into the culture supernatant using Promega's CytoTox 96® Non-Radioactive Cytotoxicity Assay Kit (Cat #G1780) according to manufacturers instructions. Target cells with either no antibody or no effector cells are used as controls to establish background levels of cell lysis.

### Example 10—Effect of Combined Imatinib and Anti-Cytokine Receptor mAb Therapy on CML Cells In Vivo

All animal studies are performed under appropriate institutional guidelines and ethics approval. Experiments are performed as previously described (Wolff N C and Ilaria R L Jr 2001 Blood 98, 2808-2816) however using primary human CML cells instead of cell lines. Human CML cells are injected into sub-lethally irradiated (300cGy) NOD/SCID or NOD/SCID/IL-2Rγnull mice (6-10 weeks old) via the tail vein. The CML cells are either Peripheral Blood Mononuclear Cells (PBMC) isolated from CML patient (1-10×10⁶ cells/mouse) or CD34+ sorted bone marrow cells from patients (0.5-5×10⁶ cells per mouse). Leukemic cell engraftment in peripheral blood is monitored by measuring human CD45+ cells by flow cytometry (Lock et al., 2002 Blood 99, 4100-4108). The tumor is allowed to establish for 2-8 weeks and then mice are treated as shown below in groups of 5-10 animals per treatment group:
1. Untreated (saline) control
2. Imatinib or other TKI (50 mg/kg every morning and 100 mg/kg every evening by gavage: Drug is administered in a volume of 250 μL sterile water by means of straight or curved animal feeding needles)
3. Imatinib or other TKI (50 mg/kg every morning and 100 mg/kg every evening by gavage: Drug is administered in a volume of 250 μL sterile water by means of straight or curved animal feeding needles) and one or more antibody selected from antibodies to CD123, CD116, CD114 and CD131 at 200-600 μg/mouse (or matched isotype control antibody at the same concentration) administered 3 times per week by intraperitoneal injection
4. An antibody to one of CD123, CD116, CD114 or CD131 at 200-600 μg/mouse (or matched isotype control antibody at the same concentration) administered 3 times per week by intraperitoneal injection

Treatment is continued for another 2-8 weeks and leukemic cell engraftment is monitored twice weekly. At the end of the treatment period mice are sacrificed and leukemic cell engraftment in peripheral blood, femoral bone marrow and spleen is determined.

In some experiments the effect of combined Imatinib and anti-cytokine receptor mAb therapy on the self renewal capacity (leukemic stem cell activity) is determined by secondary transplant experiments. In these experiments CML cells are isolated from the bone marrow of primary recipient mice treated as above (two femurs and two tibias per mouse) and secondary transplantion is performed by intravenous transplantation of 2-10×10⁶ CML cells per secondary recipient mouse (4-10 mice per group). Level of engraftment in BM and spleen of secondary recipients is measured 4-12 weeks post transplantation.

To examine the efficacy of these agents in a minimal residual disease setting animals are treated with Imatinib alone (or other TKI alone) to induce a minimal residual disease response prior to commencement of the combination therapy some experiments are conducted as follows: Imatinib (or other TKI) treatment is initiated (as above) and continued for 2-6 weeks before commencement of mAb treatments (as above) with or without continuation of Imatinib treatment in primary recipient mice. Leukemic cell engraftment is monitored in peripheral blood over the course of the experiment and at the end of the treatment period mice are sacrificed and leukemic cell engraftment in peripheral blood, femoral bone marrow and spleen is determined. Residual leukemic stem cell activity at the end of this treatment is also measured by secondary transplantation experiments conducted as outlined above.

Many modifications will be apparent to those skilled in the art without departing from the scope of the present invention.

1. A method for the treatment of Ph+ leukemia in a patient, said method comprising administering to the patient (i) a BCR-ABL tyrosine kinase inhibitor, and (ii) an agent which selectively binds to a cell surface receptor expressed on Ph+ leukemic stem cells, wherein the receptor is selected from the group consisting of IL-3Rα, G-CSFR, GM-CSFRα and the beta-common receptor for IL-3 and GM-CSF.

2. The method according to claim 1, wherein the tyrosine kinase inhibitor is imatinib.

3. The method according to claim 1, wherein the tyrosine kinase inhibitor is not imatinib.

4. The method according to claim 3, wherein the tyrosine kinase inhibitor is selected from the group consisting of dasatinib, nilotinib, bosutinib, axitinib, cediranib, crizotinib, damnacanthal, gefitinib, lapatinib, lestaurtinib, neratinib, semaxanib, sunitinib, toceranib, tyrphostins, vandetanib, vatalanib, INNO-406, AP24534, XL228, PHA-739358, MK-0457, SGX393 and DC2036.

5. The method according to claim 4, wherein the tyrosine kinase inhibitor is selected from the group consisting of dasatinib and nilotinib.

6. The method according to claim 1, wherein the agent is an antigen binding molecule.

7. The method according to claim 6, wherein the antigen binding molecule is a monoclonal antibody, or an antigen-binding and/or variable-domain-comprising fragment thereof.

8. The method according to claim 7, wherein the antigen binding molecule is a monoclonal antibody which binds selectively to IL-3Rα.

9. The method according to claim 6, wherein the antigen binding molecule comprises a modified Fc region with enhanced effector function.

10. The method according to claim 9, wherein the enhanced effector function is antibody dependent cell mediated cytotoxicity.

11. The method according to claim 6, wherein a cytotoxic compound is conjugated to the antigen binding molecule.

12. The method according to claim 1, wherein the agent is a mutein selected from the group consisting of IL-3 muteins, G-CSF muteins and GM-CSF muteins, wherein the mutein selectively binds to a receptor selected from the group consisting of IL-3R, G-CSFR, GM-CSFR but does not lead to signal activation.

13. The method according to claim 12, wherein the mutein is an IL-3 mutein.

14. The method according to claim 12, wherein a cytotoxic compound is conjugated to the mutein.

15. The method according to claim 1, wherein the agent is a soluble receptor which is capable of binding to IL-3.

16. The method according to claim 15, wherein the agent is an extracellular portion of IL-3Rα or a fusion polypeptide comprising an extracellular portion of IL-3Rα fused to an extracellular portion of common β-chain.

17. The method according to claim 1, wherein the patient is a human.

18. The method according to claim 1, wherein the Ph+ leukemia is selected from chronic myeloid leukemia (CML), acute lymphoid leukemia (ALL) and acute myeloid leukemia (AML).

19. The method according to claim 18, wherein the Ph+ leukemia is CML.

20. The method according to claim 1, wherein the BCR-ABL tyrosine kinase inhibitor is administered to the patient until the patient enters remission at which time the agent which selectively binds to a cell surface receptor expressed on Ph+ leukemic stem cells is added to the therapy.
